(19) United States
(12) Patent Application Publication
(10) Pub. No.: US 2010/0076200 A1
(43) Pub. Date: Mar. 25, 2010

(54) Purification of olmesartan medoxomil

(76) Inventors: Lilach Hedvati, Doar Na Hefer (IL); Gideon Pilarsky, Holon (IL); Yuriy Raizi, Natanya (IL); Esti Esty Marom, Ramat Gan (IL)

Correspondence Address: KENYON & KENYON LLP, ONE BROADWAY, NEW YORK, NY, 10004, US

(21) Appl. No.: 12/586,186

(22) Filed: Sep. 17, 2009

Related U.S. Application Data
(62) Division of application No. 11/217,352, filed on Sep. 2, 2005.
(60) Provisional application No. 60/638,736, filed on Dec. 22, 2004.
(60) Provisional application No. 60/606,437, filed on Sep. 2, 2004.

(51) Int. Cl. C07D 405/14

(52) U.S. Cl. 548/253

(57) ABSTRACT

The present invention provides a process for purifying olmesartan medoxomil.

This application claims the benefit of U.S. Provisional Patent Application Ser. Nos. 60/606,437 filed Sep. 2, 2004 and 60/638,736 filed Dec. 22, 2004.

### FIELD OF INVENTION

The present invention relates to processes for purifying olmesartan medoxomil.

### BACKGROUND OF THE INVENTION

The chemical name for olmesartan medoxomil is 4-(1-hydroxy-1-methylethyl)-2-propyl-1-[[2′-(1H-tetrazol-5-yl)[1,1′-biphenyl]-4-yl]methyl]-1H-imidazole-5-carboxylic acid (5-methyl-2-oxo-1,3-dioxol-4-yl)methyl ester (Merck Index 13th ed.).

The chemical structure of olmesartan medoxomil is:

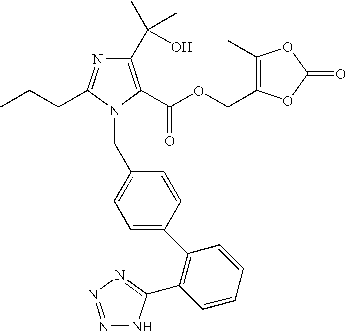

The empirical formula is C₂₉H₃₀N₆O₆.

The molecular weight is 558.58.

Olmesartan medoxomil is a prodrug that is hydrolyzed during absorption, and it is a selective AT₁ subtype angiotensin II receptor antagonist. Olmesartan medoxomil is disclosed by U.S. Pat. No. 5,616,599 to Yanagisawa et al. It is marketed as BENICAR® in film-coated tablets of 5 mg, 20 mg, and 40 mg for treatment of hypertension in a human.

The synthesis of olmesartan medoxomil (OLM-Mod) per se is illustrated as follows (see also Annu. Rep. Sankyo Res. Lab 2003, 55, 1-91):

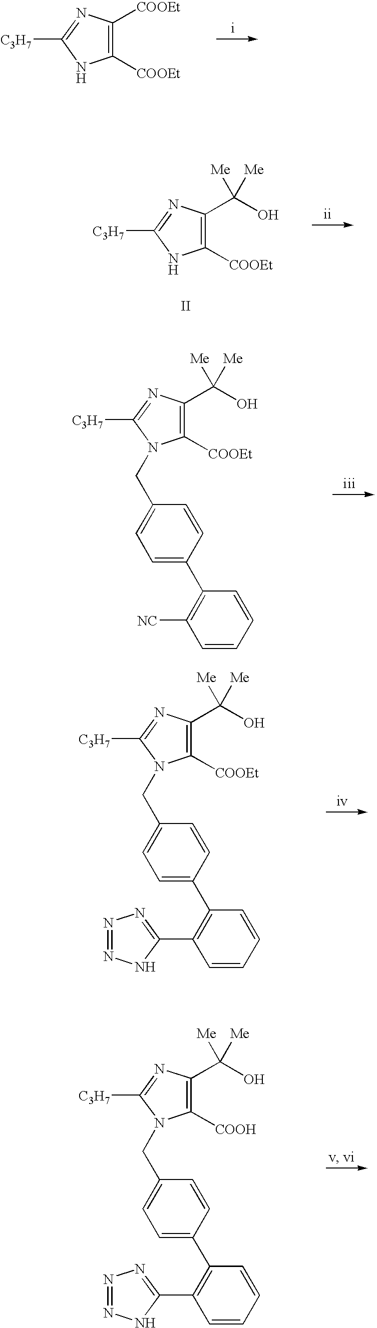

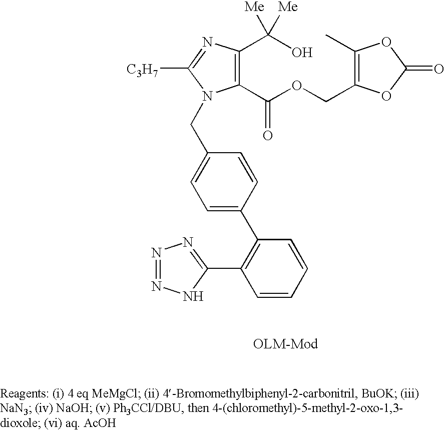

The prior art synthetic methods focus on the coupling between the substituted imidazole and the substituted biphenyl methylene bromide. Additional synthetic methods for these olmesartan medoxomil intermediates are described by: JP 11302260, JP 11292851, JP 07053489, JP 06298683, U.S. Pat. No. 5,621,134, EP 838458, DE 19757995, U.S. Pat. No. 6,111,114, and U.S. Pat. No. 6,214,999.

Step (vi) (the deprotection step) of the prior art synthesis is illustrated as follows:

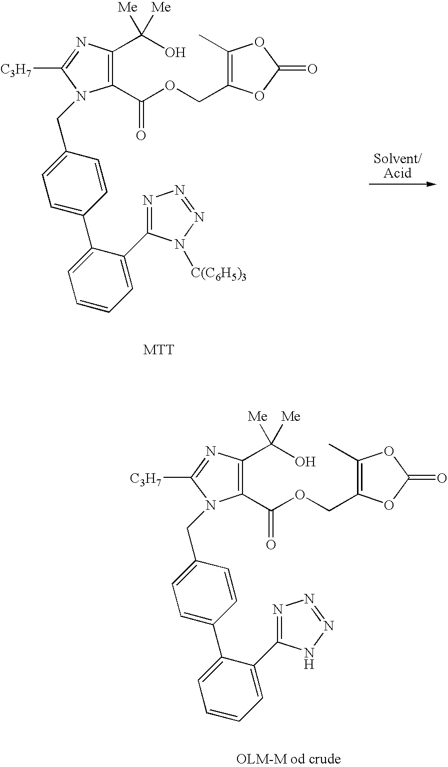

Example 61(b) of the '599 patent discloses a process for preparing crude olmesartan medoxomil from a mixture of trityl olmesartan medoxomil (MTT) and aqueous acetic acid. Col. 176, lines 24-37. Triphenyl carbinol (TPC) is removed, and olmesartan medoxomil is isolated by evaporation.

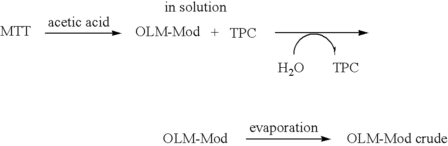

Because of the acidic conditions and the presence of water, the impurity OLM-acid is also formed during the reaction by hydrolysis of the ester bond. The chemical structure of OLM-acid is:

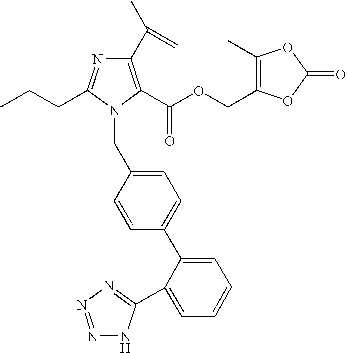

The empirical formula of OLM-acid is C₂₄H₂₆N₆O₃, and its molecular weight is 446.50. The prior art process yields crude olmesartan medoxomil containing 2.2% OLM-acid per area percent HPLC. The '599 patent also discloses that the compounds can be further purified by conventional means including recrystallization. Col. 64, lines 43-45. BENICAR® contains 0.3% OLM-acid per area percent HPLC.

There is a need for processes that minimize the hydrolysis of the ester bond in olmesartan medoxomil and thus provide a purer product. Further, from an industrial and practical viewpoint, it would be desirable to avoid the need for chromatographic purification steps. The present invention provides improved processes for purifying olmesartan medoxomil as well as olmesartan medoxomil with low levels of OLM-acid.

### BRIEF DESCRIPTION OF THE FIGURES

FIG. 1 depicts a typical chromatogram for a purified olmesartan medoxomil sample.

### SUMMARY OF THE INVENTION

In one aspect, the present invention provides a process for purifying olmesartan medoxomil including the steps of: providing a solution of olmesartan medoxomil in a C₃₋₆ ketone, preferably acetone; adding water to the solution; and recovering purified olmesartan medoxomil. The process can further include the step of heating the solution. The process can further include the step of cooling the solution after adding water to precipitate purified olmesartan medoxomil.

In another aspect, the present invention provides a process for preparing olmesartan medoxomil including the steps of: contacting trityl olmesartan medoxomil with an acid in a water miscible organic solvent, with or without water, preferably acetone and water, to obtain a first solution of olmesartan medoxomil and a precipitate of triphenyl carbinol; separating the precipitate of triphenyl carbinol from the first solution; contacting the first solution with a base to obtain a precipitate of olmesartan medoxomil; recovering the precipitate of olmesartan medoxomil; dissolving the precipitate of olmesartan medoxomil in a C₃₋₆ ketone, preferably acetone, to form a second solution; adding water to the second solution; and recovering the purified olmesartan medoxomil.

In another aspect, the present invention provides olmesartan medoxomil containing less than about 0.3% OLM-acid, more preferably less than about 0.05%, and most preferably less than about 0.03%. The present invention also provides pharmaceutical compositions containing such olmesartan medoxomil.

### DETAILED DESCRIPTION OF THE INVENTION

In one embodiment, the present invention provides a process for purifying olmesartan medoxomil including the steps of: providing a solution of olmesartan medoxomil in a C₃₋₆ ketone; adding water to the solution; and recovering purified olmesartan medoxomil.

Preferably, the C₃₋₆ ketone is acetone, methyl ethyl ketone, diethyl ketone, or t-butyl methyl ketone. Most preferably, the C₃₋₆ ketone is acetone.

For providing a solution of olmesartan medoxomil in the C₃₋₆ ketone, a preferable amount of the ketone is at least about 7 volumes ketone to about 1 gram of solid olmesartan medoxomil, more preferably at least about 10 volumes ketone to about 1 gram of solid olmesartan medoxomil. The ketone can contain water, such as about 4% to about 14% water by volume, preferably about 4% water by volume.

The process can further include the step of heating the solution of olmesartan medoxomil in the C₃₋₆ ketone. In this embodiment, the solution of olmesartan medoxomil in the C₃₋₆ ketone is preferably heated to about 30° C. to about reflux temperature, more preferably about 40° C. to about reflux temperature.

Water is added to precipitate the purified olmesartan medoxomil. The amount of water added is preferably about 0.5 to about 2 volumes water to about 1 volume of the C₃₋₆ ketone, more preferably at least about 1:1 by volume.

After adding the water, the process can further include a step of cooling the solution to induce precipitation. The solution can be cooled to a temperature below about 30° C., more preferably to about room temperature. As used herein, the term “room temperature” refers to a temperature of about 20° C. to about 30° C., preferably about 20° C. to about 25° C.

Recovering the purified olmesartan medoxomil can be performed by any means known in the art, such as filtration or centrifugation. The process can further include the step of drying the precipitated purified olmesartan medoxomil. Drying may be carried out, for example, by heating to a temperature of about 30° C. to about 60° C. The pressure can be reduced to accelerate the drying process, for example, to below one atmosphere, more preferably to below about 100 mm Hg.

In another embodiment, the present invention provides a process for preparing olmesartan medoxomil including the steps of: contacting trityl olmesartan medoxomil with an acid in a water miscible organic solvent to obtain a first solution of olmesartan medoxomil and a precipitate of triphenyl carbinol; separating the precipitate of triphenyl carbinol from the first solution; contacting the first solution with a base to obtain a precipitate of olmesartan medoxomil; recovering the precipitate of olmesartan medoxomil; dissolving the precipitate of olmesartan medoxomil in a C₃₋₆ ketone to form a second solution; adding water to the second solution; and recovering the purified olmesartan medoxomil.

Preferred water miscible organic solvents include, but are not limited to, acetone, acetonitrile, and t-butanol. Acetone is especially preferred. Preferably, the trityl olmesartan medoxomil is contacted with a mixture of a water miscible organic solvent and water. Most preferably, the trityl olmesartan medoxomil is contacted with a mixture of acetone and water. Preferably, the ratio of water to the water miscible organic solvent, e.g., acetone, is preferably about 1:3 to about 3:1 by volume.

The acid that is contacted with the first solution removes the triphenyl carbinol to form an acid salt of olmesartan medoxomil. Preferably, the acid is a strong acid having a pH of about 0 to about 4. Suitable acids include, but are not limited to, organic acids such as formic acid, acetic acid, benzoic acid, and oxalic acid; oxoacids such as perchloric acid, chloric acid, chlorous acid, hypochlorous acid, sulfuric acid, sulfurous acid, p-toluene sulfonic acid, nitric acid, nitrous acid, phosphoric acid, and carbonic acid; and binary acids such as hydrofluoric acid, hydrochloric acid, hydrobromic acid, hydrocyanic acid, and hydrosulfuric acid. Hydrochloric acid, p-toluene sulfonic acid, and especially sulfuric acid are preferred. Preferably, the amount of acid is about 2 to about 8 equivalents, more preferably about 3 to about 4 equivalents, and most preferably about 3 equivalents.

When contacting the trityl olmesartan medoxomil with the acid, the temperature is preferably about 10° C. to about 60° C., more preferably about 40° C. In a preferred embodiment, the combination of trityl olmesartan medoxomil, the water miscible organic solvent, and the acid is maintained for about 3 to about 15 hours. Preferably, the combination is maintained for about 4 to about 6 hours, most preferably for about 4 hours.

In a preferred embodiment, prior to separating the triphenyl carbinol, water is added to avoid the formation of undesired by-products. Preferably, the amount of added water is about 2 volumes per gram of trityl olmesartan medoxomil. Precipitation can be perceived visually as a clouding of the solution or formation of distinct particles of the precipitate suspended in the solution or collected at the bottom the vessel containing the solution.

Separating the triphenyl carbinol from the solution can be performed by any means known in the art, such as filtration or centrifugation.

After separating the triphenyl carbinol, the olmesartan medoxomil solution is contacted with a base. Suitable bases include, but are not limited to, alkali and alkaline earth metal hydroxides, carbonates, and hydrogen carbonate salts. Specific exemplary bases include, but are not limited to, sodium hydroxide, potassium hydroxide, calcium hydroxide, magnesium hydroxide, sodium carbonate, potassium carbonate, sodium bicarbonate, potassium bicarbonate, and calcium carbonate. Potassium carbonate and especially sodium bicarbonate are preferred. Preferably, the equivalents of base used is about equal to the equivalents of acid used, that is, the amount of base used is preferably about 0.8 to 1.5 equivalents compared to the amount of acid used. The base preferably increases the pH of the solution, but the solution need not reach a basic pH. After contacting the solution with the base, the solution is preferably maintained at a temperature of about 2° C. to about 25° C., preferably at about room temperature. As used herein, the term “room temperature” refers to a temperature of about 20° C. to 30° C., preferably 20° C. to 25° C. The solution is maintained until olmesartan medoxomil is precipitated.

The precipitate, i.e., the crude olmesartan medoxomil, can then be recovered by any means known in the art, such as filtration or centrifugation. Olmesartan medoxomil is recovered in its free base form, i.e., the nitrogen on the tetrazole is free.

The reaction progress can be detected by any method known in the art, such as, for example, HPLC, GC, TLC, NMR, and mass spectroscopy.

The processes of the present invention yield olmesartan medoxomil having low levels of OLM-acid. All percentages of impurities described herein are provided as area percentage HPLC at 220 nm. Crude olmesartan medoxomil prepared according to U.S. Pat. No. 5,616,599 contains 2.2% OLM-acid. In contrast, crude olmesartan medoxomil prepared according to the present invention contains less than about 1% OLM-acid, e.g., only about 0.89% OLM-acid.

With respect to a purified product, BENICAR® contains 0.3% OLM-acid. Thus, the prior art process reduces the OLM-acid from 2.2% in the crude olmesartan medoxomil to 0.3%. However, when such crude olmesartan medoxomil is purified according to the process of the present invention, the amount of OLM-acid is reduced to 0.26%. The amount of OLM-acid can be further reduced by utilizing crude olmesartan medoxomil prepared according to the present invention. When crude olmesartan medoxomil contains less than about 1% OLM-acid, the purification process of the present invention can reduce the OLM-acid level to less than about 0.3%.

The present invention further provides olmesartan medoxomil having less than about 0.3% OLM-acid, more preferably less than about 0.05%, and most preferably less than about 0.03%. The present invention also provides pharmaceutical compositions containing such olmesartan medoxomil.

Pharmaceutical compositions containing the olmesartan medoxomil as described above can be prepared as medicaments to be administered orally, parenterally, rectally, transdermally, bucally, or nasally. Suitable forms for oral administration include solid forms such as tablets, powders, granulates, capsules, suppositories, sachets, troches, and lozenges, as well as liquid forms such as syrups, suspensions, and elixirs. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

In addition to the active ingredient(s), the compositions of the present invention can contain one or more excipients or adjuvants. An excipient is an inert ingredient added to a pharmaceutical composition to dilute it or to give it form or consistency. An adjuvant assists the action of an active ingredient. Selection of excipients and adjuvants and the amounts to use can be readily determined by the formulation scientist based upon experience and consideration of standard procedures and reference works in the field.

Diluents increase the bulk of a solid pharmaceutical composition, and can make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure, e.g., from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be colored using any pharmaceutically acceptable colorant to improve their appearance and/or facilitate patient identification of the product and unit dosage level.

In liquid pharmaceutical compositions of the present invention, the active ingredient and any other solid excipients are suspended in a liquid carrier such as water, vegetable oil, alcohol, polyethylene glycol, propylene glycol, or glycerin.

Liquid pharmaceutical compositions can contain emulsifying agents to uniformly disperse the active ingredient(s) and/or insoluble excipient(s) throughout the composition. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxy toluene, butylated hydroxyanisole, and ethylenediamine tetraacetic acid can be added at levels safe for ingestion to improve storage stability.

According to the present invention, a liquid composition can also contain a buffer such as gluconic acid, lactic acid, citric acid or acetic acid, sodium gluconate, sodium lactate, sodium citrate or sodium acetate.

The dosage form of the present invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

Although the most suitable administration in any given case will depend on the nature and severity of the condition being treated, the most preferred route of the present invention is oral. The dosages can be conveniently presented in unit dosage form and prepared by any of the methods well-known in the pharmaceutical arts.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica.

A capsule filling of the present invention can comprise any of the aforementioned blends and granulates that were described with reference to tableting, however, they are not subjected to a final tableting step.

### EXAMPLES

### Example 1

### Preparation of Olmesartan Medoxomil

A 250 round bottom flask was charged with MTT (10 g), acetone/water (2/2 vol.), and 3 eq of H₂SO₄. The combination was stirred at room temperature for about 4-6 hrs. Triphenyl carbinol (TPC) was precipitated by adding water and filtered out. NaHCO₃ was added to the filtrate, and the mixture was cooled to 5° C. and stirred for 1 hr. Crude olmesartan medoxomil was obtained as white crystals (90% yield, OLM-acid: 0.89% area by HPLC).

### Example 2

### Purification (Crystallization) of Olmesartan Medoxomil

A 1 L flask was charged with acetone. Crude olmesartan medoxomil was added, and the mixture was heated to reflux for 1 hr and concentrated to 10 volumes. The solution was cooled to room temperature, and water (10 vol) was added. The mixture was stirred for 1 hr at room temperature, and the precipitate was filtered and dried at 45° C. under 10 mm Hg (yield 87%). The OLM-acid content was 0.04%, as determined by HPLC.

### Example 3

### Purification (Crystallization) of Olmesartan Medoxomil

A 1 L flask was charged with acetone containing 4% water by volume. Crude olmesartan medoxomil (10 g) was added, and the mixture was heated to reflux for 1 hr. The solution was cooled to room temperature, and water (10 vol) was added. The mixture was stirred for 1 hr, and the precipitate was filtered and dried at 45° C. under 10 mm Hg (yield 90%). The OLM-acid content was 0.04%, as determined by HPLC.

### Example 4

### Purification (Crystallization) of Olmesartan Medoxomil

A 1 L flask was charged with acetone containing 4% water by volume. Crude olmesartan medoxomil (10 g) was added, and the mixture was heated to reflux for 1 hr. The solution was cooled to room temperature, and water (10 vol) was added. The mixture was stirred for 1 hr at 2° C., and the precipitate was filtered. The solid white powder was dried at 45° C. under 10 mm Hg (yield 95%). The OLM-acid content was 0.07%, as determined by HPLC.

### Example 5

### Purification (Crystallization) of Olmesartan Medoxomil

A slurry of olmesartan medoxomil in acetone (7.5 vol) was heated to reflux for 1.5 hr. The mixture was cooled to room temperature, and water (10 vol) was added. The mixture was stirred for 1 hr at room temperature, and the precipitate was filtered and dried at 45° C. under 10 nun Hg (yield 91%). The OLM-acid content was 0.06%, as determined by HPLC.

### Example 6

### Impurity Profile Determination of Olmesartan Medoxomil

A 0.1% olmesartan medoxomil standard solution was prepared by diluting 15 mg of olmesartan medoxomil standard in a 50 ml volumetric flask to volume with diluent. This solution was diluted 1/50 and then 1/20 with diluent.

An olmesartan medoxomil sample solution was prepared by diluting 15 mg of olmesartan medoxomil sample in a 50 ml volumetric flask to volume with diluent.

The standard solutions were injected with a stop time of 20 minutes.

The sample solutions were injected continuing the chromatogram up to the end of gradient.

HPLC

| Column & packing | Discovery HS C18 50 * 4.6 mm, 3μ C.N | | |
|---|---|---|---|
| | 269250-U | | |
| Eluent A: | 0.025 M NaClO₄ adjusted to pH = 2.5 with | | |
| | HClO₄ | | |
| Eluent B: | Acetonitrile | | |
| | | Eluent | |
| | Time (min) | A (%) | Eluent B (%) |
| Gradient of Eluent: | 0 | 75 | 25 |
| | 15 | 55 | 45 |
| | 25 | 35 | 65 |
| | 30 | 35 | 65 |

| | |
|---|---|
| Stop time: | 30 min |
| Equilibration time: | 5 min |
| Flow: | 1.5 ml/min |
| Detector: | 220 nm |
| Injection volume: | 10 μl |
| Diluent | 50% Eluent A: 50% Eluent B |
| Column temperature | 25° C. |
| Autosampler temperature | 5° C. |

The area of each impurity was determined using suitable integrator. The detection limit in the HPLC method of the OLM-acid is 0.01%.

### Calculations

$$\% Any impurity = \frac{Area imp smp \times Conc \cdot OLM std \times Potency OLM std}{Conc \cdot smp \times Area OLM std}$$

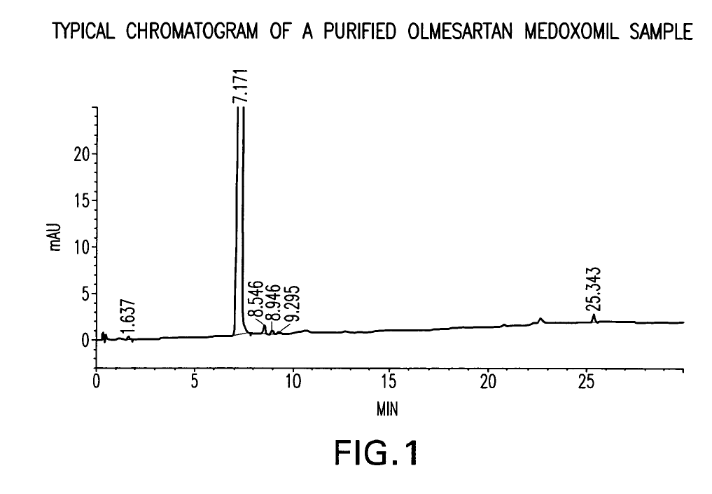

The relative retention times for the chromatographic analysis (see FIG. 1) are as follows:

| Substance | RT | RRT |
|---|---|---|
| OLM-Acid | 1.67 | 0.23 |
| OLM | 7.20 | 1.00 |
| OLM-Methyl | 8.66 | 1.20 |
| OLM-Cl | 9.05 | 1.26 |
| OLM-Eliminate | 9.41 | 1.31 |
| TPC | 18.61 | 2.58 |
| MTT | 25.37 | 3.52 |

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to, limit its scope in any way. The examples do not include detailed descriptions of conventional methods.

1. A process for purifying olmesartan medoxomil comprising:
a) providing a solution of olmesartan medoxomil in a C₃₋₆ ketone;
b) adding water to the solution; and
c) recovering purified olmesartan medoxomil.

2. The process of claim 1, wherein the C₃₋₆ ketone is selected from the group consisting of acetone, methyl ethyl ketone, diethyl ketone, and t-butyl methyl ketone.

3. The process of claim 2, wherein the C₃₋₆ ketone is acetone.

4. The process of claim 1, wherein the amount of the C₃₋₆ ketone is at least about 7 volumes to about 1 gram of solid olmesartan medoxomil.

5. The process of claim 4, wherein the amount of the C₃₋₆ ketone is at least about 10 volumes to about 1 gram of solid olmesartan medoxomil.

6. The process of claim 1, wherein the solution of olmesartan medoxomil in the C₃₋₆ ketone further comprises about 4% to about 14% water by volume.

7. The process of claim 6, wherein the solution of olmesartan medoxomil in the C₃₋₆ ketone further comprises about 4% water by volume.

8. The process of claim 1, further comprising heating the solution of olmesartan medoxomil in acetone to about 30° C. to about reflux temperature.

9. The process of claim 8, wherein the solution of olmesartan medoxomil in acetone is heated to about 40° C. to about reflux temperature.

10. The process of claim 1, wherein the amount of water added is about 0.5 to about 2 volumes water to about 1 volume of the C₃₋₆ ketone.

11. The process of claim 10, wherein the amount of water added is at least about 1 volume water to about 1 volume of the C₃₋₆ ketone.

12. The process of claim 1, further comprising cooling the solution after step b) to a temperature below about 30° C.

13. The process of claim 12, wherein the solution is cooled to about room temperature.

14. The process of claim 1, further comprising drying the purified olmesartan medoxomil.

15. The process of claim 1, wherein the purified olmesartan medoxomil contains less than about 0.3% OLM-acid.

16. The process of claim 15, wherein the purified olmesartan medoxomil contains less than about 0.05% OLM-acid.

17. The process of claim 16, wherein the purified olmesartan medoxomil contains less than about 0.03% OLM-acid.

18.-29. (canceled)
